(19) United States
(12) Patent Application Publication
(10) Pub. No.: US 2024/0407865 A1
(43) Pub. Date: Dec. 12, 2024

(54) DETERMINING INFORMATION ABOUT A SURGICAL PORT IN A SURGICAL ROBOTIC SYSTEM

(71) Applicant: Covidien LP, Mansfield, MA (US)

(72) Inventors: Barret R. Daniels, Cambridge, MA (US); Andrew W. Dennis, Golden, CO (US); Dustin C. Eastman, Longmont, CO (US)

(21) Appl. No.: 18/702,368

(22) PCT Filed: Nov. 14, 2022
(86) PCT No.: PCT/IB2022/060943

§ 371 Date: Apr. 18, 2024

Related U.S. Application Data
(60) Provisional application No. 63/280,311, filed on Nov. 17, 2021.

(51) Int. Cl. A61B 34/30; A61B 17/00; A61B 17/34; A61B 18/00; A61B 18/14; A61B 90/98

(52) U.S. Cl. CPC A61B 34/30; A61B 18/1445; A61B 18/1482; A61B 90/98; A61B 2017/00119; A61B 17/3423; A61B 2018/0063; A61B 2034/302; A61B 2560/0276

(57) ABSTRACT

A surgical robotic system for use in a minimally invasive surgical procedure includes a trocar for facilitating passage of a surgical instrument into a body cavity. The surgical trocar has a memory having stored therein information about the surgical trocar, and a conductive coil configured to inductively couple to another conductive coil in a surgical robotic arm, whereby the conductive coil of the surgical trocar communicates the information about the surgical trocar to the conductive coil of the surgical robotic arm.

### FIELD

The present technology is generally related to surgical robotic systems used in minimally invasive medical procedures.

### BACKGROUND

Some surgical robotic systems include a console supporting a surgical robotic arm and a surgical instrument or at least one end effector (e.g., forceps or a grasping tool) mounted to the robotic arm. The robotic arm provides mechanical power to the surgical instrument for its operation and movement. Each robotic arm may include an instrument drive unit operatively connected to the surgical instrument and coupled to the robotic arm via a rail. In operation, the robotic arm is moved to a position over a patient and then guides the surgical instrument into a small incision via an endoscopic port/surgical trocar or a natural orifice of a patient to position the end effector at a work site within the patient's body.

The endoscopic port may be attached to an end of the surgical robotic arm and held in a fixed position during insertion of the surgical instrument therethrough. An endoscopic port of a particular size (e.g., length and diameter) may be selected based on the patient and/or surgical procedure to be performed.

It would be advantageous to provide an improved means for determining information about an endoscopic port attached to the surgical robotic arm.

### SUMMARY

In one aspect of the disclosure, a surgical robotic system is provided and includes a surgical robotic arm and a surgical port. The surgical robotic arm includes an elongated rail, a pair of jaws attached to an end portion of the elongated rail, and a first coil configured to receive an electric current. The surgical port includes a head configured to be grasped by the pair of jaws of the surgical robotic arm, a cannula extending distally from the head and configured to receive a surgical instrument, a memory having stored therein information about the surgical port, and a second coil in communication with the memory. The second coil is configured to be inductively coupled to the first coil of the surgical robotic arm whereby the information about the surgical port is transferred from the second coil to the first coil.

In aspects, the second coil may extend around an opening defined by the head.

In aspects, the head may include a collar defining an annular recess therein configured to be grasped by the pair of jaws. The second coil may be attached to the collar.

In aspects, the first coil may be attached to one or both of the pair of jaws.

In aspects, the information about the surgical port may include a length of the cannula thereof and a diameter of the cannula thereof.

In aspects, the surgical robotic system may further include a computer configured to receive, from the first coil, the information.

In aspects, the computer may be configured to issue a visual or audible warning upon determining that the surgical port is incompatible with a surgical instrument of the surgical robotic system.

In aspects, the information about the surgical port may include calibration settings for the surgical port or an insertion depth of a surgical instrument through the surgical port.

In aspects, the surgical port may include a plurality of LEDs in communication with the second coil such that the LEDs are configured to be powered by the inductive coupling between the first and second coils.

In aspects, the surgical robotic system may be configured to illuminate the LEDs in response to detecting a fault with the surgical port.

In aspects, a plurality of surgical ports may be provided. The cannula of each of the surgical ports may have a discrete length and diameter.

In accordance with another aspect of the disclosure, a plurality of surgical ports of a surgical robotic system for insertion into a body cavity is provided. Each of the surgical ports includes a head defining an opening configured for receipt of a surgical instrument, a cannula extending distally from the head and defining a channel configured for passage of the surgical instrument, a memory having stored therein information about the surgical port, and a coil in communication with the memory and configured to be inductively coupled to another coil of the surgical robotic system.

In aspects, the cannula of each surgical port may have a discrete length and diameter. The information about each surgical port may include the discrete length and diameter of the cannula.

In aspects, the coil of each surgical port may extend around the opening defined by the head.

In aspects, the head of each surgical port may include a collar defining an annular recess therein configured to be grasped by a pair of jaws of the surgical robotic system. The coil may be attached to the collar.

In aspects, the cannula of each surgical port may include a plurality of LEDs in communication with the coil such that the plurality of LEDs are configured to be powered by the inductive coupling between the coil of the surgical port and the coil of the surgical robotic system.

In accordance with another aspect of the disclosure, a method of transmitting information about a surgical port attached to a surgical robotic arm is provided. The method includes inductively coupling a first coil of a surgical robotic arm and a second coil of a surgical port; and transferring information about the surgical port from a memory of the surgical port to the first coil via the second coil.

In aspects, the information about the surgical port may include calibration settings for the surgical port or a predetermined insertion depth of a surgical instrument through the surgical port.

In aspects, the method may further include adjusting a depth of entry of the surgical instrument through the surgical port into an access opening in a patient based on the predetermined insertion depth.

In aspects, inductively coupling the first and second coils may include passing an electric current through the first coil, whereby the first coil induces a voltage in the second coil.

In aspects, the method may further include detecting a fault with the surgical port; and illuminating a plurality of LEDs of the surgical port in response to detecting the fault.

In aspects, the information may include a length and/or a diameter of a cannula of the surgical port.

Further details and aspects of exemplary aspects of the disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the disclosure are described herein with reference to the accompanying drawings, wherein:

FIG. 1 is a schematic illustration of a surgical robotic system including a control tower, a console, and one or more surgical robotic arms;

FIG. 2 is a perspective view of a surgical robotic arm of the surgical robotic system of FIG. 1;

FIG. 3 is a perspective view of a setup arm with the surgical robotic arm of the surgical robotic system of FIG. 1;

FIG. 4 is a schematic diagram of a computer architecture of the surgical robotic system of FIG. 1;

FIG. 5 is a perspective view illustrating a surgical trocar of the surgical robotic system of FIG. 3;

FIG. 6 is a front view illustrating a head of the trocar of FIG. 5;

FIG. 7 is a top view illustrating the head of the trocar of FIG. 6 and a schematic illustration of jaws of the surgical robotic arm grasping the head; and

FIG. 8 is a flow chart illustrating an exemplary method of utilizing the surgical robotic system of FIG. 1.

### DETAILED DESCRIPTION

Embodiments of the disclosed surgical robotic system are described in detail with reference to the drawings, in which like reference numerals designate identical or corresponding elements in each of the several views. As used herein the term “distal” refers to that portion of the surgical robotic system or component thereof, that is closer to a patient, while the term “proximal” refers to that portion of the surgical robotic system or component thereof, that is further from the patient.

Surgical robotic systems need to be compatible with different sized endoscopic/laparoscopic “ports” or trocars. Longer ports allow procedures in larger patients and larger diameter ports enable the use of larger diameter tools such as a stapler. The robotic system should have knowledge of the diameter and length of the port being used to properly calibrate the instrument and maintain a proper remote center of motion in the patient's abdominal wall. During surgery, a sterile field should be maintained between the robotic arm of the robotic system and the attached port, which penetrates the abdominal wall.

Accordingly, it would be advantageous to provide a way of intelligently detecting the type of port used without compromising the sterile field. The identification of the port could provide increased patient safety by preventing insertion of instruments or endoscopes that are not compatible with the attached port. Additionally, the identification could inform the robotic system at what insertion point to maintain the remote center of motion. Using electrical connections between the robotic arm and the port are problematic because it is difficult to maintain sterility while making proper electrical contact.

Accordingly, this disclosure describes a robotic system including a robotic arm and a surgical trocar or “port” with each incorporating a coil. The port also has a non-volatile storage device having stored therein identifying information about the port (e.g., the length and diameter of the port) such that when the port is attached to the robotic arm, the robotic system inductively couples the coils to transfer information stored on the storage device of the port to the robotic system. Transferring the identifying information via an inductive coupling is efficient, accurate, and does not compromise sterility between the port and the remainder of the robotic system. Further, the inductive coupling may provide power to the port to read the information from the storage device of the port. In aspects, the robotic system may also write information onto the port. For example, the robotic system may track a usage count of the port to ensure that the port is only used once.

In another aspect, a near field technology device, such as, for example, radio-frequency identification (“RFID”), may be utilized to identify the port being used. More specifically, the port may have an RFID tag and the robotic arm (or another suitable component of the surgical robotic system) may be equipped with an RFID reader. A user may swipe the RFID tag of the port on the RFID reader to transfer the identifying information about the port from the port to the robotic system. In aspects, the robotic system may write information onto the RFID tag of the port.

In further aspects, LEDs or other suitable illumination devices may be positioned along a length of an exterior surface of the port. The robotic system may be configured to activate the LEDs, via the inductive coupling, when a safety check is triggered, thereby prompting a clinician to identify and inspect the port for any issues. For example, the clinician may be prompted to inspect the port from inside the patient using an endoscope or visually inspect the outside of the port. Issues with the port that may require examination using the safety check include chipping of the port or when a robotic joint slips and the port may have been displaced.

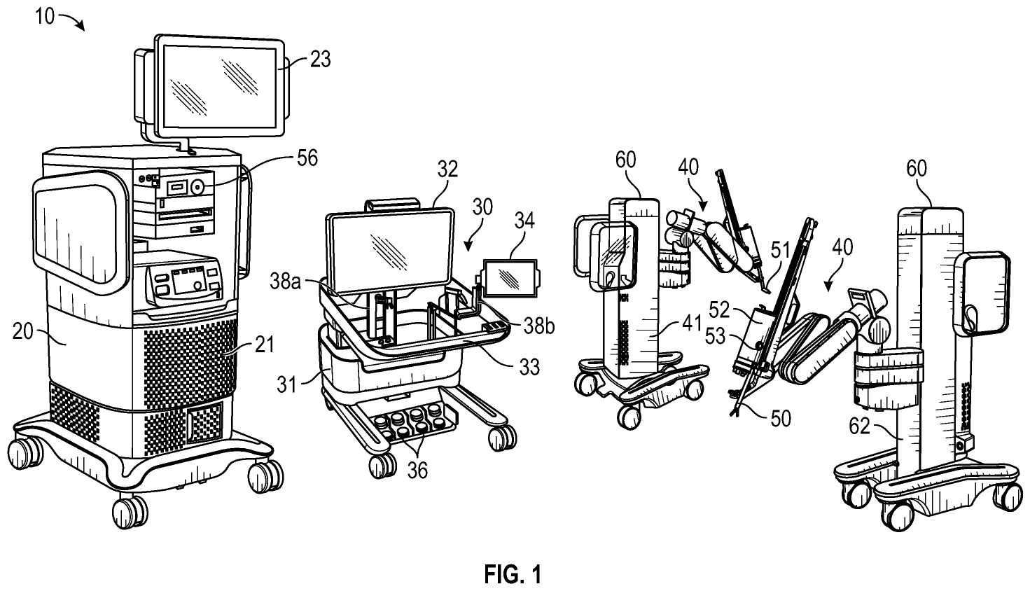

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the disclosure and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

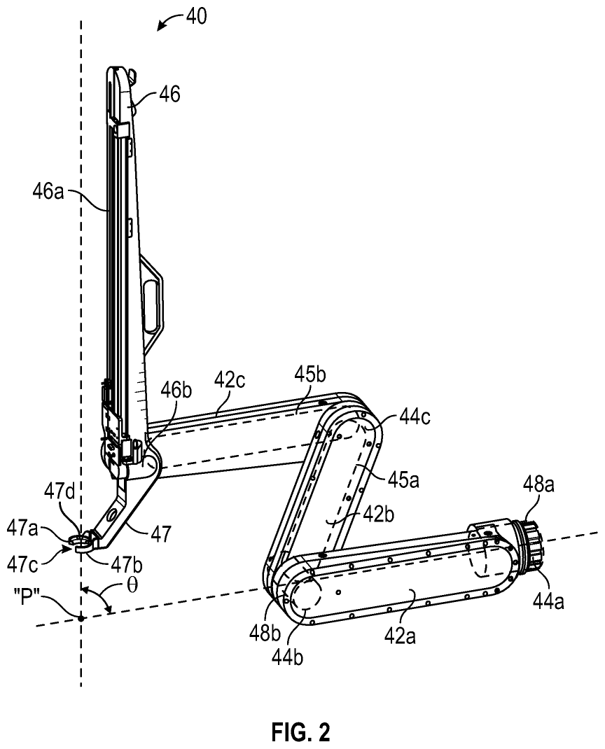

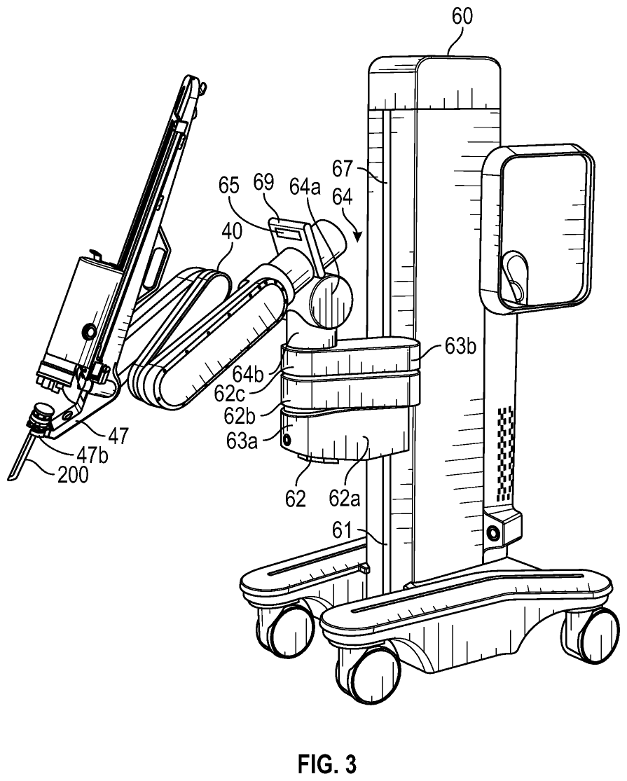

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the rail 46 relative to each other. More specifically, links 42b, 42c, and the rail 46 are passively coupled to the actuator 48b which enforces rotation about a pivot point “P” which lies at an intersection of the first axis defined by the link 42a and the second axis defined by the rail 46. Thus, the actuator 48b controls the angle θ between the first and second axes allowing for orientation of the surgical instrument 50. Due to the interlinking of the links 42a, 42b, 42c, and the rail 46 via the belts 45a and 45b, the angles between the links 42a, 42b, 42c, and the rail 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the robotic arm 40 also includes a holder, such as, for example, an elongated rail 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The rail 46 includes a sliding mechanism 46a, which is configured to move the IDU 52 along the second longitudinal axis defined by the rail 46. The rail 46 also includes a joint 46b, which rotates the rail 46 relative to the link 42c. During endoscopic procedures, the instrument 50 may be inserted through an endoscopic port or surgical trocar 200 (FIG. 3) held by the rail 46.

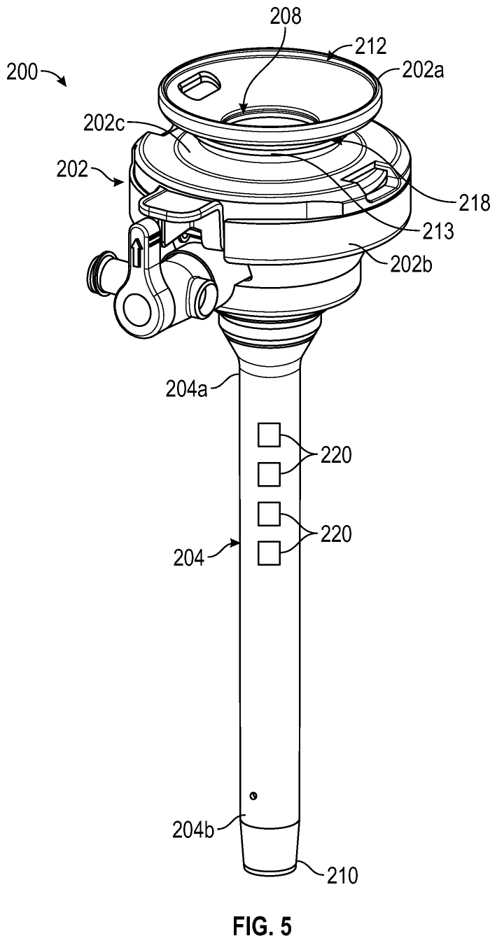

The rail 46 has a distal end portion or arm 47 disposed distally of the joint 46b. The arm 47 has a pair of arcuate jaws 47a, 47b disposed at a distal end thereof. The pair of jaws 47a, 47b define an annular opening 47c therebetween and are configured to move relative to one another between an expanded position, in which the surgical trocar 200 is positioned therebetween, and an approximated position in which the jaws 47a, 47b grasp the surgical trocar 200 to secure the surgical trocar 200 to the robotic arm 40. The rail 46 has a conductive wire, such as, for example, a coil 47d (FIGS. 2 and 7) disposed within or on the jaws 47a, 47b and positioned about the annular opening 47c. In aspects, the coil 47d may be attached to other suitable locations of the robotic arm 40 or at a location between the jaws 47a, 47b. The conductive coil 47d is in communication with the computer 41 and is supplied with an electric current from a power source, such as, for example, a battery (not shown) of the robotic system 1 or an external AC power source. The conductive coil 47d is configured to be inductively coupled to a corresponding conductive wire or coil 213 (FIG. 5) of the surgical trocar 200 to induce the surgical trocar 200 to transfer information therefrom, as will be described in detail with reference to FIGS. 5-8. The pitch, length, and diameter of the coils 47d, 213 may be selected to adjust the strength of the inductive coupling between the coils 47d, 213. In aspects, the coils 47d, 213 may be arcuate or linear along their length. To increase the strength of the inductive coupling, the coils 47d, 213 may be positioned coaxially with one another upon loading the surgical trocar 200 between the jaws 47a, 47b.

The robotic arm 40 also includes a plurality of manual override buttons 53 disposed on the IDU 52 and the setup arm 62, which may be used in a manual mode. The user may press one or more of the buttons 53 to move the component associated with the button 53.

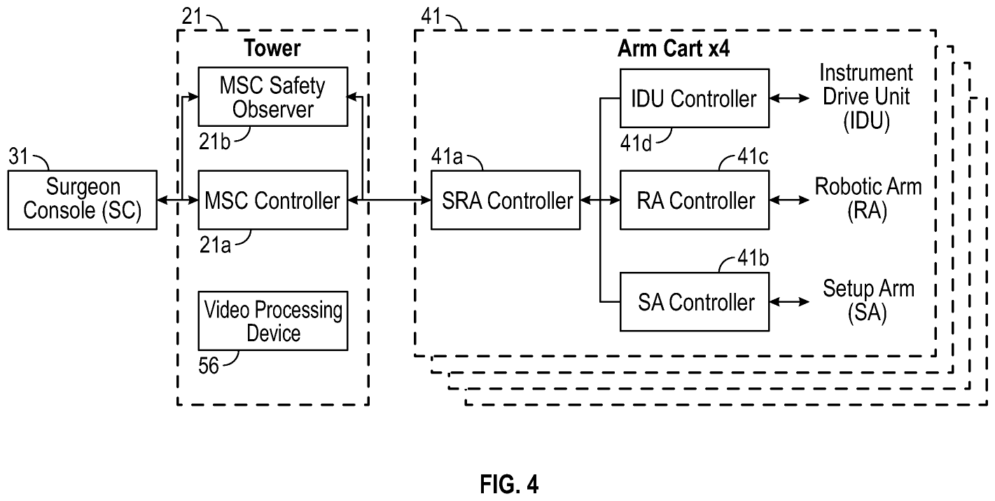

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In embodiments, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

With reference to FIGS. 5-7, the surgical trocar or port 200 of the surgical robotic system 10 is configured for guiding the surgical instrument 50 through a natural or artificial opening in a patient and into a surgical site or internal body cavity of the patient. The trocar 200 generally includes a head 202, and a cannula 204 extending distally from the head 202. The head 202 and cannula 204 collectively define a channel 208 longitudinally therethrough configured to receive the surgical instrument 50. The cannula 204 has a proximal end portion 204a monolithically formed with or otherwise attached to the head 202, and a distal end portion 204b having a distal tip 210 defining a distal port therein. The distal tip 210 may be set at an oblique angle relative to a longitudinal axis of the cannula 204 and may be sharp for traumatic insertion of the trocar 200 into tissue.

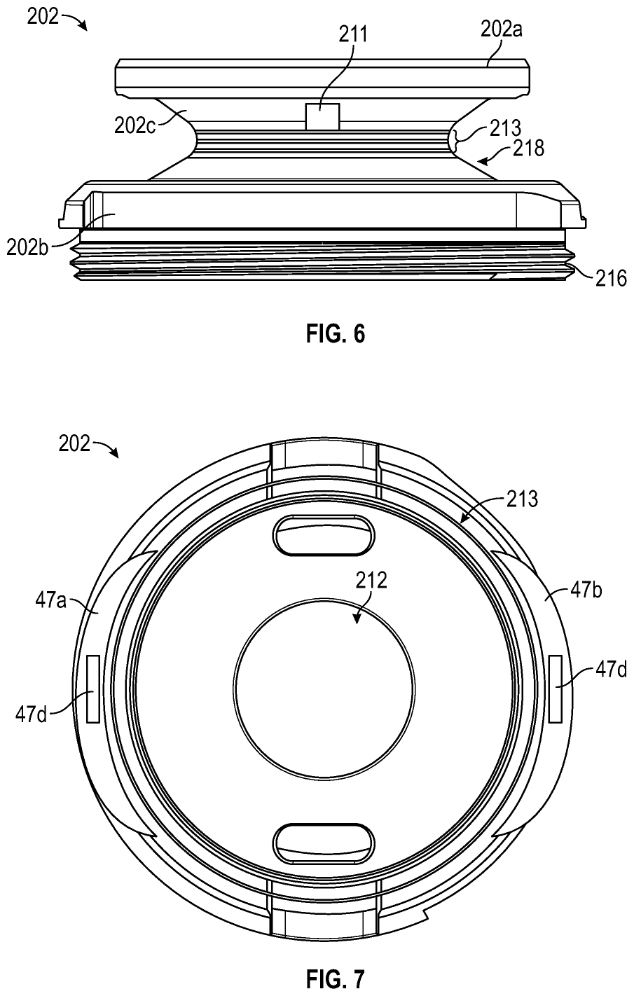

The head 202 of the trocar 200 includes a proximal lid 202a, a distal cap 202b, and a collar 202c disposed between the proximal lid 202a and the distal cap 202b. The proximal lid 202a defines an entrance opening 212 into the head 202, and the distal cap 202b has threading 216 (FIG. 6) configured to detachably, threadedly couple the head 202 to the proximal end portion 204a of the cannula 204. The collar 202c may have a substantially hourglass shape and defines an annular recess 218 therein. The annular recess 218 of the collar 202c is configured to be grasped by the pair of jaws 47a, 47b of the rail 46 (FIGS. 2 and 3).

The trocar 200 further includes a memory 211 (FIG. 6) and a conductive wire, such as, for example, a conductive coil 213, each attached to the collar 202c at the annular recess 218. The coil 213 may extend around the opening 212 defined by the head 202. In aspects, the coil 213 may be attached to other suitable locations of the trocar 200 to strengthen the inductive coupling between the coils 47d, 213. For example, the coils 47d, 213 may be positioned at their respective locations on the slide 46 and the surgical trocar 200 such that the coils 47d, 213 are coaxial upon the surgical trocar 200 being attached to the rail 46.

The memory 211 may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The memory 211 has stored therein information about the trocar 200, such as, for example, an overall length of the trocar 200, a length of the cannula 204, an inner diameter of the cannula 204, an outer diameter of the cannula 204, a weight of the trocar 200, zero rotation location, or other information about the trocar 200. The information stored in the memory 211 may additionally or alternatively include a brand of the surgical trocar 200, calibration settings of the trocar 200, a usage count of the trocar 200, life of the trocar 200, construction materials of the trocar 200, etc. In aspects, the information stored on the memory 2211 may additionally or alternatively include parameters to configure the surgical robotic system 10. As such, a newly launched trocar 200 might not need a software update of the system 10 to support its usage since the trocar 200 may be configured to indicate to the system 10 the trocar's 200 configuration parameters, such as, for example, its physical characteristics such as length, diameter, zero rotation location, weight and calibration parameters. The information stored on the memory 211 may be used by the system 10 to dynamically configure safety checks for properly installed trocars 200 and/or the trocar 200 and instrument 50 combinations.

The memory 211 is in communication (e.g., electrical contact) with the coil 213 and configured to send the information to the coil 47d of the jaws 47a, 47d of the slide 46 (FIGS. 2 and 3) in response to the coil 47d of the jaws 47a, 47b of the slide 46 (FIGS. 2 and 3) being inductively coupled to the coil 213 of the trocar 200.

The trocar 200 may further include a plurality of illumination devices or light sources 220 (FIG. 5) disposed along a length of the cannula 204 and in communication with the coil 213 of the trocar 200 such that the inductive coupling that provides power to the coil 213 also powers the light sources 220. The light sources 220 may be disposed in an annular array about any suitable portion of the cannula 204 and/or the head 202. The light sources 220 may be light-emitting diodes (LEDs) for illuminating the fields of view. In aspects, white light LEDs or other colors of LEDs or any combination of LEDs may be used, such as, for example, red, green, blue, infrared, near infrared and ultraviolet or any other suitable LED.

In aspects, the surgical robotic system 10 may include a plurality of surgical ports 200 with each of the ports 200 having the same or similar structure but having a cannula 204 with a discrete length and diameter such that each surgical port 200 is suited for a particularly sized patient and/or procedure.

In operation, a particular type of surgical port 200 (e.g., a surgical port of a particular length and diameter) is selected based on a particular procedure and/or patient and coupled to the surgical robotic arm 40. The jaws 47a, 47b of the slide 46 are approximated about the collar 202c of the head 202 of the trocar 200 to secure the head 202 of the trocar 200 to the surgical robotic arm 40, as shown in FIG. 3. Upon closing the jaws 47a, 47b about the head 202 of the trocar 200, the coil 47d disposed within the jaws 47a, 47b move into proximity with the coil 213 in the collar 202c of the surgical trocar 200.

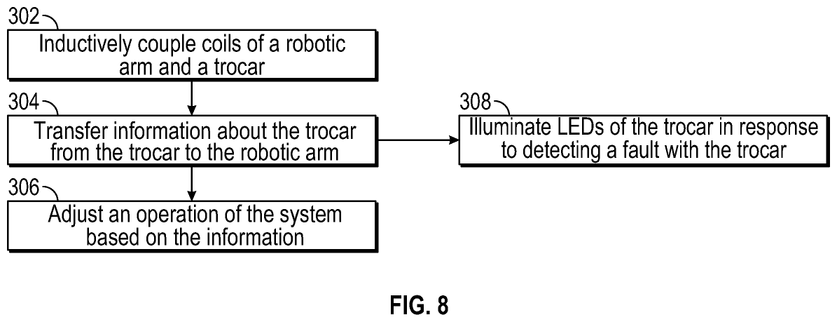

FIG. 8 illustrates a flow chart depicting an exemplary method of identifying the type of trocar 200 attached to the slide 46 of the surgical robotic system 10. In step 302 of the method, the coil 47d of the slide 46 inductively couples to the coil 213 of the trocar 200. More specifically, with the coils 47d, 213 in proximity to one another, the surgical robotic system 10 passes an electric current through the coil 47d of the slide 46, whereby the coil 47d of the slide 46 induces a voltage in the coil 213 of the trocar 200.

In step 304 of the method, with the coils 47d, 213 inductively coupled to one another, the information about the selected trocar 200 (e.g., the length and diameter of the cannula 204, calibration settings of the trocar 200, brand of the trocar 200, use life of the trocar 200, etc.) is automatically transferred from the memory 211 of the trocar 200 to the coil 47d of the slide 46 via the coil 213 of the trocar 200. The computer 41 of the surgical robotic system 10 receives the information about the trocar 200. In step 306 of the method, an operation or use of the system 10 is adjusted based on the information of the trocar 200 received by the computer 41 from the coil 47d.

Adjusting the operation or use of the system 10 includes adjusting a depth of entry of the surgical instrument 50 into an access opening in a patient. Additionally or alternatively, the computer 41 may utilize the information to prevent the use of surgical instruments or endoscopes that are inappropriate (e.g., too large) for the selected trocar 200. For example, when an instrument 50 is attached to the instrument drive unit 52, a predetermined diameter of the instrument 50 is compared to the acceptable diameters for use with the surgical trocar 200. If the diameter of the instrument 50 is too large for the surgical trocar 200, an error message or warning may be activated (e.g., prompt on the surgeon console 30, activate the LEDs 220 throughout the system, etc.) The system 10 may prevent power from being delivered to the instrument 10 if it is determined that the instrument 50 and trocar 200 are incompatible. If compatibility is found, the system 10 may provide some visual or audible confirmation to proceed with a surgical procedure.

In further aspects, when the information stored on the surgical trocar 200 includes calibration settings, adjusting the use of the system 10 may include performing a calibration of the surgical trocar 200 and/or instrument 10 by the system 10 based on the calibration settings.

In step 308 of the method, a fault with the surgical trocar 200 may be detected by the surgical robotic system 10 whereby the LEDs 220 of the surgical trocar 200 may be illuminated to warn a clinician of the fault condition. The fault of the surgical port 200 may be damage to the surgical trocar 200, slippage of the surgical trocar 200 from the slide 46, or the depth of the surgical trocar 200 being outside of a predetermined range. Since the LEDs 220 are in electrical communication with the coil 213 of the surgical trocar 200 (e.g., via a wired connection), the voltage induced in the coil 213 of the surgical trocar 200 via the inductive coupling with the coil 47d of the robotic arm 40 powers the LEDs 220.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical robotic system, comprising:
a surgical robotic arm including:
an elongated rail;
a pair of jaws attached to an end portion of the elongated rail; and
a first coil configured to receive an electric current; and
at least one surgical port including:
a head configured to be grasped by the pair of jaws of the surgical robotic arm;
a cannula extending distally from the head and configured to receive a surgical instrument;
a memory having stored therein information about the at least one surgical port; and
a second coil in communication with the memory and configured to be inductively coupled to the first coil of the surgical robotic arm whereby the information about the at least one surgical port is transferred from the second coil to the first coil.

2. The surgical robotic system according to claim 1, wherein the second coil extends around an opening defined by the head.

3. The surgical robotic system according to claim 2, wherein the head includes a collar defining an annular recess therein configured to be grasped by the pair of jaws, the second coil being attached to the collar.

4. The surgical robotic system according to claim 3, wherein the first coil is attached to at least one of the pair of jaws.

5. The surgical robotic system according to claim 1, wherein the information about the at least one surgical port includes a length of the cannula of the at least one surgical port and a diameter of the cannula of the at least one surgical port.

6. The surgical robotic system according to claim 1, further comprising a computer configured to receive, from the first coil, the information about the at least one surgical port.

7. The surgical robotic system according to claim 6, wherein the computer is configured to issue a visual or audible warning upon determining that the surgical port is incompatible with a surgical instrument of the surgical robotic system.

8. The surgical robotic system according to claim 1, wherein the information about the at least one surgical port includes calibration settings for the at least one surgical port or an insertion depth of a surgical instrument through the at least one surgical port.

9. The surgical robotic system according to claim 1, wherein the at least one surgical port includes a plurality of LEDs in communication with the second coil such that the plurality of LEDs are configured to be powered by the inductive coupling between the first and second coils.

10. The surgical robotic system according to claim 7, wherein the surgical robotic system is configured to illuminate the plurality of LEDs in response to detecting a fault with the at least one surgical port.

11. The surgical robotic system according to claim 1, wherein the at least one surgical port includes a plurality of surgical ports, the cannula of each surgical port of the plurality of surgical ports having a discrete length and diameter.

12. A plurality of surgical ports of a surgical robotic system for insertion into a body cavity, each surgical port of the plurality of surgical ports comprising:
a head defining an opening configured for receipt of a surgical instrument;
a cannula extending distally from the head and defining a channel configured for passage of the surgical instrument;
a memory having stored therein information about the surgical port; and
a coil in communication with the memory and configured to be inductively coupled to another coil of the surgical robotic system.

13. The plurality of surgical ports according to claim 12, wherein the cannula of each surgical port of the plurality of surgical ports has a discrete length and diameter, the information about each surgical port including the discrete length and diameter of the cannula.

14. The surgical robotic system according to claim 10, wherein the cannula of each surgical port of the plurality of surgical ports includes a plurality of LEDs in communication with the coil such that the plurality of LEDs are configured to be powered by the inductive coupling between the coil of the surgical port and the another coil of the surgical robotic system.

15. A method of transmitting information about a surgical port attached to a surgical robotic arm, the method comprising:
inductively coupling a first coil of a surgical robotic arm and a second coil of a surgical port; and
transferring information about the surgical port from a memory of the surgical port to the first coil via the second coil.

16. The method according to claim 15, wherein the information about the surgical port includes at least one of calibration settings for the surgical port or a predetermined insertion depth of a surgical instrument through the surgical port.

17. The method according to claim 16, further comprising adjusting a depth of entry of the surgical instrument through the surgical port into an access opening in a patient based on the predetermined insertion depth.

18. The method according to claim 15, wherein inductively coupling the first and second coils includes passing an electric current through the first coil, whereby the first coil induces a voltage in the second coil.

19. The method according to claim 15, further comprising:
detecting a fault with the surgical port; and
illuminating a plurality of LEDs of the surgical port in response to detecting the fault.

20. The method according to claim 15, wherein the information includes at least one of a length or a diameter of a cannula of the surgical port.
